(19) United States
(12) Patent Application Publication
(10) Pub. No.: US 2011/0034706 A1
(43) Pub. Date: Feb. 10, 2011

(54) PROCESS FOR PREPARING N-VINYLPYRROLIDONE FROM LACTONE-FREE PYRROLIDONE

(75) Inventors: Wolfgang Staffel, Waldsee (DE); Stefan Kaeshammer, Schifferstadt (DE); Roland Kessinger, Weinheim (DE); Regina Vogelsang, Ludwigshafen (DE); Axel Paul, Lampertheim (DE); Lembit Tuttelberg, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/745,604

(22) PCT Filed: Nov. 26, 2008
(86) PCT No.: PCT/EP2008/066251

§ 371 Date: Oct. 27, 2010

(30) Foreign Application Priority Data
Dec. 7, 2007 (EP) 07122636.9

(51) Int. Cl. C07D 207/267

(52) U.S. Cl. 548/552

(57) ABSTRACT

A process for preparing N-vinylpyrrolidone by reacting 2-pyrrolidone with acetylene, wherein the 2-pyrrolidone used as a starting material (referred to hereinafter as starting 2-pyrrolidone) comprises less than 1 part by weight of γ-butyrolactone per 100 parts by weight of 2-pyrrolidone.

The invention relates to a process for preparing N-vinylpyrrolidone by reacting 2-pyrrolidone with acetylene, wherein the 2-pyrrolidone used as a starting material (referred to hereinafter as starting 2-pyrrolidone) comprises less than 1 part by weight of γ-butyrolactone per 100 parts by weight of 2-pyrrolidone.

N-Vinylpyrrolidone is prepared industrially by vinylating 2-pyrrolidone with acetylene. 2-Pyrrolidone (γ-butyrolactam) in turn is obtainable by reacting butyrolactone with ammonia, as described, for example, in DE-A 1 795 007. The 2-pyrrolidone used in the preparation of N-vinylpyrrolidone therefore generally still comprises residual amounts of γ-butyrolactone.

It was an object of the present invention to provide a process for preparing N-vinyl-pyrrolidone with high space-time yields and maximum yields of N-vinylpyrrolidone.

Accordingly, the process defined at the outset has been found.

The starting materials for the process are 2-pyrrolidone and acetylene.

The 2-pyrrolidone used in the process according to the invention is referred to hereinafter as starting 2-pyrrolidone.

2-Pyrrolidone is also referred to as γ-butyrolactam and is the known compound of the formula

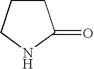

According to the invention, the starting 2-pyrrolidone used is a starting material which comprises less than 1 part by weight, preferably less than 0.5 part by weight and more preferably less than 0.3, especially less than 0.15 and most preferably less than 0.1 part by weight of γ-butyrolactone of the formula

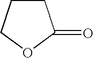

per 100 parts by weight of 2-pyrrolidone.

γ-Butyrolactone can be removed subsequently from 2-pyrrolidone to the desired degree. Alternatively, 2-pyrrolidone can actually be prepared such that the 2-pyrrolidone obtained does not comprise more than the amounts of γ-butyrolactone specified above. 2-Pyrrolidone with the maximum contents of γ-butyrolactone specified above is available on the market.

Suitable starting 2-pyrrolidones may comprise other impurities or by-products; they preferably comprise them, if at all, only in small amounts.

In particular, suitable starting 2-pyrrolidone, per 100 parts by weight of 2-pyrrolidone, may, if appropriate, have the following further constituents:

from 0 to 2 parts by weight, especially from 0 to 1 part by weight, more preferably from 0 to 0.1 part by weight of water and

from 0 to 2 parts by weight, especially 0 to 1 part by weight, more preferably from 0 to 0.1 part by weight of methylpyrrolidone.

The starting 2-pyrrolidone consists of 2-pyrrolidone preferably to an extent of more than 97% by weight, especially to an extent of more than 98% by weight, more preferably to an extent of more than 98.5% by weight and even more preferably to an extent of more than 99% by weight, especially to an extent of more than 99.5% by weight or to an extent of more than 99.7% by weight.

The acetylene used may also, if appropriate, comprise by-products and impurities. In particular, suitable acetylene may, if appropriate, comprise, per 100 parts by weight of acetylene, also up to 2 parts by weight, especially up to 1 part by weight, of propyne.

The starting 2-pyrrolidone is reacted with acetylene preferably in the presence of a catalyst.

Useful catalysts have been found to be especially alkali metal pyrrolidates.

For this purpose, starting 2-pyrrolidone is preferably first reacted with an alkali metal hydroxide or alkali metal alkoxide. It may, for example, be lithium hydroxide, sodium hydroxide or potassium hydroxide; particular preference is given to potassium hydroxide.

The alkali metal hydroxide is preferably used in the form of an aqueous solution. The content of alkali metal hydroxide may, for example, be from 5 to 90% by weight, based on the solution; in particular, it is from 30 to 60% by weight, more preferably from 45 to 55% by weight.

The reaction with the alkali metal hydroxide is effected preferably at temperatures of from 50 to 250° C. and from 1 mbar to 1 bar, especially at from 20 to 250° C. The temperature at the top of the column is preferably from 20 to 100° C., especially from 25 to 60° C.; the temperature in the bottom of the column is preferably from 100 to 250° C., especially from 120 to 200° C.

This reaction is preferably conducted semicontinuously or continuously. Particular preference is given to conducting it continuously.

Preference is given to effecting the reaction in a column, more preferably in a column with random packing or structured packing, which is operated at the aforementioned temperatures and pressures. It is preferably operated continuously.

Particular preference is given to columns which comprise both random packings and structured packings, for example comprise beds of random packings in the lower section and have packing elements (for example installed steel sheets) in the upper section.

The column has preferably at least two, more preferably at least 3 theoretical plates. It may have, for example, from 2 to 100, especially from 3 to 20 theoretical plates.

The alkali metal hydroxide and 2-pyrrolidone are preferably added to the column in the upper third, more preferably in the upper quarter, of the column.

The mean residence time of the alkali metal hydroxide and of the starting 2-pyrrolidone in the reaction zone, i.e. column, is less than 6 minutes, especially less than 5 minutes; more preferably, it is from 50 to 200 seconds.

The reaction forms the corresponding alkali metal salt, i.e. the alkali metal pyrrolidate, preferably potassium pyrrolidate.

The amount of alkali metal hydroxide is preferably selected such that from 0.25 to 25% by weight, preferably from 5 to 20% by weight, of the 2-pyrrolidone is present as the pyrrolidate, i.e. potassium pyrrolidate.

The product of the reaction can be drawn off continuously in the lower part of the column or at the bottom of the column. Subsequently, the compound is then reacted with acetylene, preferably in a separate reaction vessel.

The alkali metal salt catalyzes the subsequent reaction with acetylene (vinylation). The effluent from the above column can be mixed with further starting 2-pyrrolidone.

The 2-pyrrolidone used for the vinylation is then present preferably to an extent of from 0.25 to 10% by weight, especially from 1.5 to 6% by weight, as the pyrrolidate.

The subsequent vinylation with acetylene can likewise be effected batchwise, semicontinuously or continuously.

The vinylation is preferably effected continuously.

The reaction with acetylene is preferably carried out at temperatures of from 120 to 220° C., more preferably at from 140 to 170° C., and preferably at pressures of from 1.0 to 25 bar and more preferably at from 10 to 20 bar.

By virtue of the process according to the invention, N-vinylpyrrolidone is obtainable in high yield and purity. In contrast, the use of starting 2-pyrrolidone which comprises more than 1 part by weight of γ-butyrolactone per 100 parts by weight of 2-pyrrolidone shows much poorer yields. The deterioration in the yield of vinylpyrrolidone goes beyond the mere content of γ-butyrolactone which is not vinylated. The presence of more than 1 part by weight of γ-butyrolactone therefore impairs the yield of vinylpyrrolidone in a nonobvious and disproportionate manner.

### EXAMPLES

### Example 1

85.1 g (1 mol) of 2-pyrrolidone and 2.05 g (30 mmol) of potassium methoxide were initially charged, and methanol was distilled off under reduced pressure. 30 g of the mixture thus obtained were subjected to vinylation at 150° C. and 20 bar of acetylene for 1 h. The yield of N-vinylpyrrolidone (NVP) was 69% of theory (determined by gas chromatography).

### Comparative Example 1

85.1 g (1 mol) of 2-pyrrolidone, 2.05 g (30 mmol) of potassium methoxide and 2.58 g (30 mmol) of gamma-butyrolactone were initially charged, and methanol was distilled off under reduced pressure. 30 g of the mixture thus obtained were subjected to vinylation at 150° C. and 20 bar of acetylene for 1 h. The yield of N-vinylpyrrolidone (NVP) was 49% of theory.

### Comparative Example 2

85.1 g (1 mol) of 2-pyrrolidone, 2.05 g (30 mmol) of potassium methoxide and 1.29 g (15 mmol) of gamma-butyrolactone were initially charged, and methanol was distilled off under reduced pressure. 30 g of the mixture thus obtained were subjected to vinylation at 150° C. and 20 bar of acetylene for 1 h. The yield of N-vinylpyrrolidone (NVP) was 52% of theory.

### Example 2

85.1 g (1 mol) of 2-pyrrolidone, 2.05 g (30 mmol) of potassium methoxide and 0.26 g (3 mmol) of gamma-butyrolactone were initially charged, and methanol was distilled off under reduced pressure. 30 g of the mixture thus obtained were subjected to vinylation at 150° C. and 20 bar of acetylene for 1 h. The yield of N-vinylpyrrolidone (NVP) was 66% of theory.

1. A process for preparing N-vinylpyrrolidone comprising reacting 2-pyrrolidone with acetylene, wherein the 2-pyrrolidone used as a starting material (hereinafter “starting 2-pyrrolidone”) comprises less than 1 part by weight of γ-butyrolactone per 100 parts by weight of 2-pyrrolidone.

2. The process according to claim 1, wherein the starting 2-pyrrolidone comprises less than 0.15 part by weight of γ-butyrolactone.

3. The process according to claim 1, wherein the reaction of the starting 2-pyrrolidone with acetylene is effected continuously at temperatures of from 120 to 220° C. and pressures of from 1 to 25 bar.

4. The process according to claim 1, wherein the reaction is effected in the presence of potassium pyrrolidate as a catalyst.
